(19) United States
(12) Patent Application Publication
(10) Pub. No.: US 2022/0272994 A1
(43) Pub. Date: Sep. 1, 2022

(54) GOODS PROTECTION INSERT AND USES THEREOF

(71) Applicant: LIVA BIO PROTECTION TECHNOLOGIES LTD, Rehovot (IL)

(72) Inventor: Ifat Hammer, Rehovot (IL)

(21) Appl. No.: 17/631,675

(22) PCT Filed: Aug. 20, 2020
(86) PCT No.: PCT/IL2020/050916

§ 371 Date: Jan. 31, 2022

(30) Foreign Application Priority Data
Aug. 22, 2019 (IL) 268855

(51) Int. Cl. A23B 7/155; C12N 1/20; B65D 81/28; A01G 24/22

(52) U.S. Cl. CPC A23B 7/155; C12N 1/20; B65D 81/28; A01G 24/22; A23V 2002/00

(57) ABSTRACT

The present disclosure provides protection inserts comprising a discrete substrate-forming material holding a nutrient composition, the nutrient composition comprising a combination of nutrients for supporting selective growth of one or more bacteriocin-producing bacterium on said substrate and being essentially free of externally added microorganisms Also disclosed herein is a consumer goods package comprising goods to be protected and a protection insert disclosed herein as well as methods of protecting goods making use of the insert disclosed herein.

### BACKGROUND ART

References considered to be relevant as background to the presently disclosed subject matter are listed below:
International application publication No. WO93/09676.
Japanese application publication No JP61,030,550.
US patent application publication No. 20150272145
U.S. Pat. No. 10,226,061
International application publication No. WO15/187638,
US patent application publication No. 2013/236603,
German patent application No. DE102010021027,
U.S. Pat. No. 7,795,000;
U.S. Pat. No. 8,038,990;
International patent application publication No. WO04/030624,
International patent application publication No. WO14/170621,
International patent application publication No. WO14/114805,
International patent application publication No. WO14/209912,
U.S. Pat. No. 8,617,625,
International patent application publication No. WO14/145369,
U.S. Pat. No. 5,989,601,
International patent application publication No. WO00/60947,
Spanish application No. ES19951101,
Belgian application No. BE1018502

Acknowledgement of the above references herein is not to be inferred as meaning that these are in any way relevant to the patentability of the presently disclosed subject matter.

### TECHNOLOGICAL FIELD

The present disclosure relates to goods protection devices and methods for use in improving quality of goods.

### BACKGROUND

The food industry in constantly seeking alternative, non-chemical food preservation methods as well as non-chemical methods for improving goods quality.

International patent application publication No. WO93/09676 discloses a method for preserving a food product, such as meat where the meat is inoculated with an effective amount of live euhygienic non-pathogenic, non-spoilage bacteria (L. delbrueckii or Hafnia alvei) in order to competitively inhibit the growth of undesired pathogenic and spoilage bacteria.

Japanese patent application publication No JP61,030,550 discloses immobilized live lactic acid bacteria (LAB) on agar or gelatin stored in a film packaging material with food.

US patent application Publication No. 20150272145 describes a method for preparing raw meat or raw seafood that is resistant to spoilage, by incubating a solution comprising a population of non-pathogenic psychrotrophic lactic acid bacteria under conditions and for a period of time sufficient to enable growth of the bacterial population to form a bacterial culture capable of preventing spoilage; adding a saccharide to the bacterial culture and incubating the bacterial culture and admixing the bacterial culture formed with the raw meat or raw seafood.

U.S. Pat. No. 10,226,061 describe the prolongation of shelf life and quality of packaged food by the use of a microbial oxygen absorber.

Other publications describing the use of live bacteria in preservation of food include International application publication No. WO15/187638, US patent application publication No. 2013/236603, German patent application No. DE102010021027, U.S. Pat. Nos. 7,795,000; 8,038,990; International patent application publication No. WO04/030624, International patent application publication No. WO14/170621, International patent application publication No. WO14/114805, International patent application publication No. WO14/209912, U.S. Pat. No. 8,617,625, International patent application publication No. WO14/145369, U.S. Pat. No. 5,989,601, International patent application publication No. WO00/60947, Spanish application No. ES19951101, Belgian application No. BE1018502.

### GENERAL DESCRIPTION

The present disclosure aims at increasing shelf-life of consumer goods and fresh food products by stimulating growth of selected bacteria within packages containing the goods, without the use of externally added live bacteria to the package, thereby eliminating the high costs and the complexity of handling live bacteria in industrial plants.

In accordance with a first of its aspects, the present disclosure provides a protection insert comprising a discrete substrate-forming material holding a nutrient composition, the nutrient composition comprising a combination of nutrients for supporting selective growth of one or more bacteriocin-producing bacterium on said substrate and being essentially free of externally added microorganisms.

In accordance with a second aspect, the present disclosure provides a package comprising consumer goods to be protected and the protection insert disclosed herein.

In accordance with a third aspect, the present disclosure provides a method of protecting consumer goods, the method comprising packaging the goods in a package holding in vicinity with said goods a protection insert disclosed herein.

Finally, in accordance with a fourth aspect, there is disclosed a method of improving crop yield, the method comprising introducing into a soil in which said crop is cultivated at least one insert disclosed herein, and allowing said crop to grow in the presence of said insert.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1B is an image of two cucumber bags after 12 days of storage, the left side bag (FIG. 1A) being stored with a protection insert of the non-limiting example of Table 2 and the right side bag (FIG. 1B) acting as negative control (stored with no insert).

### DETAILED DESCRIPTION OF EMBODIMENTS

The current disclosure is based on the understanding of the probiotic bacteria ability to compete with pathogens and produce antimicrobial bacteriocins. Yet, instead of adding live bacteria cells as hitherto described, natural growth of bacteria inherently present in the surrounding environment is, according to the present disclosure, stimulated by using a relative cheap blend of nutrients which selectively promotes the growth of specifically desired bacteria.

Bacteriocins are potent antimicrobial peptides produced inter alia by lactic acid bacteria (LAB) or by other bacteria such as Bacillus species and are mainly active against Gram-positive bacteria. Their production is sensitive to both the microbial strain and the culture conditions. Several isolated, purified or excreted bacteriocins are industrially used in food preservation and few are also FDA-approved. They are considered to be safe as they are readily degraded by the human gastrointestinal tract and are commonly used in food products for fermentation purposes or for increasing nutritional value.

Thus, in accordance with the present disclosure, there is provided nutrient blends selective for the growth of bacteriocin producing bacteria, inter alia, for food preservation/protection or for supporting crop/plant growth and yield. Specifically, the present disclosure provides, in accordance with a first of its aspects, a food protection and/or preservation insert comprising a discrete substrate-forming material holding a nutrient composition, the nutrient composition comprising a combination of nutrients for supporting selective growth of one or more bacteriocin-producing bacterium on said substrate and being essentially free of externally added microorganisms.

In the context of the present disclosure when referring to a “discrete substrate forming material” it is to be understood to refer to a distinct solid or semi-solid body that can be physically held, visualized, or moved and is not physically connected to the goods with which it is held.

In the context of the present disclosure when referring to “essentially free of externally added microorganisms” it is to be understood that no microorganisms were added to the insert and the microorganisms found on the insert are only those grown during protection and/or preservation period or the growing period (e.g. when used for supporting plant growth). In other words, the insert before use, e.g. before packing with the goods to be preserved should be free of detectable microorganisms.

In the context of the present disclosure, the term “protection insert” denotes a unit form that is used to protect consumer goods from spoiling as a result of microbial growth of pathogenic or otherwise undesired bacteria on the goods. The goods may be any edible material, including industrially produced food stuff, fresh produce, crops (also during their cultivation/growth period), etc., as well as non-edible goods, that have a tendency to spoil/rotten, such as cosmetics., home care products and drugs.

All the above is collectively being referred to herein as “goods”.

The protection insert is designed such to selectively support the growth of human friendly bacteriocin-producing bacterium that is naturally present in the environment (in the air, i.e. airborne bacteria, in the soil, on the goods itself). Without being bound by theory, it is believed that such bacteriocin producing bacterium, selectively grown on the insert, and its spread inside on or in the surrounding of the goods (either through water vapors or biofilm formation) allows for the preservation of quality of the goods by competing with other spoiling or pathogenic bacteria and secreting antimicrobial bacteriocins.

In the context of the present disclosure, a “bacteriocin-producing bacterium” or “bacteriocin producing bacteria” denotes any bacterium or consortium of bacteria that produce peptides with anti-microbial activity via their metabolism. For simplicity, in the following, when referring to bacteriocin producing bacterium it is to be understood as also encompassing a consortium of two or more such bacteria.

In some examples, the bacteriocin producing bacterium comprises lactic acid bacteria (LAB). Such bacteria are known to have the ability to inhibit growth of undesired microorganism, including, inter alia, spoilage microorganisms and pathogenic bacteria. This inhibitory activity is the result of the metabolic product secreted by these LAB which acts as antimicrobial compounds. These compounds include organic acids, diacetyl, hydrogen peroxide and bacteriocin.

In some other examples, the bacteriocin producing bacterium comprises a member of the Bacillus species.

In some examples, the protection insert can be utilized for preventing growth of pathogenic microorganism and/or spoilage microorganism in packed goods, thereby increasing the shelf-life of the goods.

In some examples, the protection insert disclosed herein extends the shelf life of a good by at least 10%, at times, by at least 20%, at times by at least 30%, at times by at least 50%, at times by at least 60%, at times by at least 70%, at times by at least 80%, at times by at least 90%, at times by even 100% as compared to the shelf life of the same goods package stored under the same conditions, without the protection insert.

In the context of the present disclosure, when referring to spoilage microorganism it is to be understood as encompassing microorganism that cause undesirable changes to the quality of goods (cause goods, such as food, to deteriorate and develop unpleasant odors, tastes, and textures, e.g. cause fruits and vegetables to get mushy or slimy, or meat to develop a bad odor) but are not toxic to consume. The spoilage microorganism typically metabolizes gases, acids, sulfur compounds and nitrogen compounds that impact on the sensorial properties of the goods, specifically when referring to food stuff.

When referring to pathogenic microorganism it is to be understood as encompassing microorganism that are harmful to consume, or they produce toxins that are harmful or fatal if consumed. Some non-limiting examples of pathogenic microorganism include Campylobacter, Salmonella, E. Coli, Clostridium Perfringens, Bacillus Cereus, Listeria Monocytogenes, Shigella spp., Staphylococcus Aureus, Streptococcus and Vibrio bacteria.

When referring to a semi solid insert it is to be understood to encompass a deformable, non-fluidic unit form, i.e. flexible, pliable, easily bent, reshapable, while when referring to a solid form it is to be understood as encompassing a rigid structure, i.e. non-flexible, that does not change in shape upon application of pressure. The physical property of the insert, namely, it being semi solid or solid (or in other words, non-liquid), depends, inter alia, on the type of substrate forming material used for holding the nutrients composition. In this connection, it is noted that while the insert as a whole is semi solid or solid, the nutrients composition therein may be in a non-solid form, such as a gel, semi-gel, liquid, fluid (e.g. powder) form. This is achieved, for example, by holding the non-solid nutrients composition on a solid or semi solid substrate material.

In the context of the present disclosure, the term “substrate forming material” denotes a food grade material that under suitable conditions forms into a matrix that is capable of holding liquids and specifically an aqueous solution in which the nutrients disclosed herein are dispersed or dissolved.

In some examples, the substrate forming material comprises or is a gel or jelly like material. In some examples, the substrate forming material is a gel or jelly like material typically used as a substrate for microbial growth.

In some examples, the substrate forming material comprises or is selected from the group consisting of agar (agar-agar), guar gum, xanthan gum, gellan gum, carrageenan, gelatin, dextrin and starch.

In one specific example, the substrate forming material is agar-agar, namely, the gelatinous (jelly like) carbohydrate material derived from marine algae, typically used as a base for bacterial culture media and as a stabilizer and thickener in many food products.

In some examples, the amount of substrate forming material is between 0.1% and 5% out of the total weight of the composition, at times, between 0.5% and 2%, at times, between 0.6% and 3%, at times, between 0.7% and 2.5, and at times, between 0.8-1.7% or any other range within the range of 0.1% and 5%.

The substrate forms, in the presence of water, a matrix or scaffold capable of holding a combination of ingredients suitable for human use, such as food grade ingredients (e.g. when the goods are food).

Notably, in the context of the present disclosure the term “food grade” denotes any material that is acceptable and approved for human consumption.

The nutrients in the nutrient composition are selected to selectively promote growth of the bacteriocin producing bacterium, the latter inhibiting or preventing, as aforementioned, growth of undesired microorganisms. In other words, in the presence of the selective nutrients held by the substrate, predominantly only the desired bacteriocin-producing bacterium is effectively grown.

In the context of the present disclosure, when referring to selectively promote growth of the bacteriocin producing bacterium it is to be understood to predominantly allow growth of only this bacterium, without a detectable amount or with less than 10 CFU/gr growth or even less than 5 CFU/gr of other microorganisms, such as yeast, enterococcus bacteria etc.

The nutrient composition held by the substrate comprises different types of substances, each acting as a source for a different nutrient element required for the growth to the bacteriocin producing bacterium.

In some examples, the nutrient composition comprises one or more nitrogen containing compounds, acting as a nitrogen source for the growth of the desired, bacteriocin-producing bacterium.

In some examples, the nitrogen containing compounds are any one or combination of amino acids, short peptides, polypeptides, and protein hydrolysates. These may be obtained from various sources.

In some examples, the source for the nitrogen containing compounds is an animal source, such as beef extract and/or casein hydrolysate.

In some examples, the source for the nitrogen containing compounds is a microorganism extract, such as yeast extract.

In some examples, the source for the nitrogen containing compounds is a plant extract, such as a legume extract. In this context, the legume may be any type of legume, such as, without being limited thereto, peas, chickpeas, potato, soy, beans, and others.

In some examples, the nitrogen containing compounds comprises a combination of one or more extracts and one or more hydrolysates from different sources.

In some examples, the nitrogen containing source comprises at least a protein hydrolysate, and at times preferably peptone.

In some examples, the nitrogen containing source comprises at least yeast extract.

In some examples, the nitrogen containing source comprises at least animal extract.

In some examples, the nitrogen containing source comprises at least plant extract.

In some examples, the nitrogen containing source comprises at least a protein hydrolysate and yeast extract.

In some examples, the nitrogen containing source comprises a combination of a protein hydrolysate, yeast extract and at least one of animal extract or plant extract.

In some examples, the amount of the nitrogen containing compounds/source, be it a single source or a combination of sources is between 0.1% and 20% out of the total weight of the insert, at times between 0.5% and 10%, at times between 0.5% and 5%, at times between 1% and 5%, at times between 1% and 3%, at times between 0.5% and 3%, at times between 0.1% and 3% or any other range within the range of 0.1% and 20%.

In some examples, the carbohydrates are a carbon containing compound acting as a carbon source.

In some examples, the carbon containing compound is or comprises saccharides, these include, inter alia, any one or combination of monosaccharides, e.g. glucose, fructose, galactose, xylose, arabinose; disaccharides e.g. sucrose, lactose, maltose, isomaltulose, trehalose, trealulose and trehalulose; and oligosaccharides i.e. those typically containing 3-10 monosaccharides, such as raffinose (trisaccharide) oligofructose (FOS), galacto-oligosaccharides (GOS), gluco-oligosaccharide, isomalto-oligosacccharides, maltotriose and others.

Other carbohydrates may include sugar alcohols such as mannitol and sorbitol,

In some examples, the carbohydrate/carbon containing compound comprises at least glucose or at least a combination of glucose and FOS.

In some examples, the amount of the carbohydrates, be it a single source or a combination of carbohydrates, is within the range of 0.5% and 5% out of the total weight of the insert composition, at times, between 1% and 5%, at times between 0.5% and 4%, at times between 0.5% and 2.5% and times between 1% and 4%, at times between 1% and 3%, at times between 1% and 2.5% or any other range within the range of 0.5% and 5%.

In some examples, the inorganic salts are minerals. These can include, without being limited thereto, salts of phosphate, potassium, calcium, zinc, magnesium, manganese, and iron.

In some examples, the source of such minerals/inorganic salts is yeast extract. Notably, the yeast extract can be regarded as providing several types of nutrients in the context of the present disclosure, including the minerals, the vitamins and/or the digested nucleic acids.

In some examples, the nutrient composition comprises at least manganese salts, such as manganese sulfate.

In some examples, the nutrient composition comprises at least magnesium salts, such as magnesium sulfate.

In some examples, the nutrient composition comprises at least sodium salt, such as sodium acetate.

In some examples, the nutrient composition comprises at least potassium salts such as dipotassium hydrogen phosphate.

In some examples, the nutrient composition comprises at least ammonium salts, such as tri-ammonium citrate.

In some examples, the nutrient composition comprises at least a combination of manganese salts and magnesium salts.

In some examples, the amount of the inorganic minerals/inorganic salts, be it a single inorganic component or a combination of inorganics, is within the range of 0.005% and 5% out of the total weight of the composition, at times between 0.01-2% and 0.02-1%, at times between 0.005% and 2%, at times between 0.05% and 1%, at times between 0.01% and 3% or any other range within the range of 0.005% and 5%.

In some examples, the nutrient composition comprises at least one surfactant/emulsifier.

In some examples, the surfactant/emulsifier is or comprises fatty acids or fatty acid esters or any other surfactant/emulsifier acceptable under the food regulations (E-numbers).

In the context of the present invention, the term “fatty acids” denotes simple fatty acids, namely, those having a carboxylic head-group and an aliphatic tail which is either saturated or unsaturated, and yet also complexed fatty acids, where the head-group is substituted with macromolecule, such as a polyoxyethylene group. Such fatty acids may be used also as surfactants and/or emulsifiers of the nutrient composition.

The aliphatic chain may include any number of carbon atoms including short chain fatty acids, having a tail of up to 5 carbon atoms, medium chain fatty acids, containing a tail of 6-12 carbons, long chain fatty acids typically including 13-21 carbon atoms in the tail, and very long chain fatty acids, containing 22 and more carbons in the aliphatic tail.

In some examples, the fatty acids component of the nutrient composition comprise one or combination of caprylic acid, capric acid, lauric acid, myristic acid, palmitic acid, stearic acid, arachidic acid, behenic acid, oleic acid, linoleic acid, polysorbate 20, polysorbate 40, polysorbate 60 and polysorbate 80.

In some further examples, the fatty acids component comprises at least polysorbate 80.

In some examples, the amount of surfactant/emulsifier, be it a single surfactant/emulsifier or a combination of several such additives, is within the range of 0.001% and 5% out of the total weight of the composition, at times between 0.005% and 3%, at times between 0.01% and 2% and times between 0.01% and 3%, at times between 0.05% and 3%, at times between 0.05% and 2%, at times between 0.01% and 1%, at times between 0.05% and 1%, at times between 0.06% and 0.11%, or any other range within the range of 0.001% and 5%.

In some cases, the nutrient composition also requires the presence of one or more vitamins. The vitamins are known to be important for proper function of organisms and metabolism and are thus essential even at very low quantities.

The nutrient composition may include, without being limited thereto, any one or combination of niacin (Vitamin B13), Calcium Pantothenate (calcium salt of vitamin B5), Pyroxidine (Vitamin B6) and Vitamin B12. In some examples, the nutrient composition comprises at least calcium panthenoate.

In some examples, the vitamin component includes at least niacin.

In some examples, the vitamin comprises a combination of niacin and calcium panthenoate.

In some examples, the amount of vitamins is within a range of 0.0001 and 5%, at times between 0.0001% and 3%, at times between 0.005% and 5%, at times between 0.008% and 2%, and any other range between 0.0001% and 5%. Notably, the vitamins can be obtained from the yeast extract and yet can also be externally added, as shown in the non-limiting examples of Table 2 below.

The nutrient composition also comprises, according to some examples, a buffering agent. The purpose of the buffering agent, inter alia, is to maintain the pH of the nutrient composition within a pH range that supports the growth of the desired bacteriocin-producing bacterium. In some examples, the buffering agent comprises any one or combination of phosphoric acid, citric acid, lactic acid and glycine.

In some examples, the buffering agent comprises at least glycine.

The amount of the buffering agent will depend on the type of the agent used. In some examples, the amount is selected to provide a pH within the range of 5.5 and 7, at times, between 5.5 and 6.5, at times, between 5.6 and 6, at times between 5.7 and 6.1, at times about 5.8±2.

The protection insert may include additional ingredients.

In some examples, the amount of the preservatives added to the nutrient composition is within the range of 0.01% and 0.1% out of the total weight of the composition, at times between 0.05% and 0.1%, at times between 0.02% and 0.08% or any other range within the range of 0.01% and 0.1%. The amount may vary, depending on the preservative used.

In some examples, the nutrient composition also comprises inactivated cell extract of LAB, this may assist in supporting the growth of LAB on the insert.

Examples of such inactivated cell extract may include but not limited to: extract of Lactobacillus Lactis, and extract of Lactobacillus Salivarius.

In yet some other or additional examples, the nutrient composition comprises one or more preservatives (growth inhibitors). Non-limiting examples of preservatives include potassium sorbate, sodium benzoate, sodium chloride, sodium lactate, bacteriocins, long chain polyphosphates, ammonium citrate and sodium acetate.

In one example the protection additive is potassium sorbate.

It is noted that while the insert is typically in a semi solid or solid form, as described above, the nutrient composition may be in liquid, fluid solid form, as long as it is held by the insert.

The protection insert disclosed herein can support the growth of various LAB. Examples of LAB include Lactobacillus Plantarum, Lactobacillus Lactis, Lactobacillus Brevis.

In some examples, the insert stimulates and/or supports the growth of bacteria other than LAB such as Bacillus species. These include, without being limited thereto, Bacillus Subtillis, Bacillus Pumilis, Bacillus Safensis, Bacillus Thurgiensis, Bacillus amyloliquefaciens, Bacillus Licheniformis, Bacillus Megaterium, Bacillus Coagulans, and Bacillus Brevis.

Without being bound to theory, the selective growth is achieved by providing the specific nutritional requirements (namely, the nutrient composition disclosed herein) specific for the desired bacteria along with generating conditions which inhibit the growth of other undesired microorganisms. Such conditions include, for example, any one or combination of preservatives, pH control agents, oxygen control and temperature control.

In some examples, the selective growth can be supported or achieved by any one or combination of controlling the oxygen level, e.g. by monitoring or specifically designing the permeability of the package holding the goods, by controlling the storage temperature, by controlling pH using specifically selected pH adjusting agents etc.

The ratio between the different components of the insert may vary depending on the good to be preserved, the conditions of storage (temperature, humidity etc.,) and the type of bacteriocin producing microorganism, the stimulation of which is desired.

The nutrient composition can be prepared by any method known in the art. in some examples, the insert is prepared by mixing the ingredients of the nutrient composition with the substrate forming material until a homogenous mixture is formed.

Tables 1A-1B provide exemplary and non-limiting ranges for some components that are included in the insert, in accordance with some examples of the present disclosure.

TABLE 1A
Beef-extract containing composition

| Ingredient | Amount (w %) |
|---|---|
| Protein hydrolysate (e.g. peptone/soy hydrolysate/casein | 0.5-10% |
| hydrolysate, gelatin hydrolysate) | |
| Beef extract | 0.5-10% |
| Yeast extract* | 0.1-2% |
| Saccharide (e.g. glucose) | 0.5-5% |
| Sodium Acetate | 0.1-2% |
| Surfactant/emulsifier (e.g. Polysorbate 80) | 0.005-3% |
| Dipotassium hydrogen phosphate | 0.005-1% |
| Ammonium citrate | 0.005-1% |
| Magnesium sulfate | 0.001-1% |
| Manganese sulfate | 0.001-1% |
| Substrate material (e.g. Agar) | 0.1-5% |
| Reverse osmosis (RO) Water | q.s |
| Total | 100.0% |

*The yeast extract provides also the vitamins

TABLE 1B
Vegan based insert composition

| Ingredient | Amount (w %) |
|---|---|
| Vegetable Peptone/Soy hydrolysate | 0.1-2% |
| Pea Hydrolysate/Potato hydrolysate/Bean Hydrolysate | |
| Yeast extract | 0.1-2% |
| Soy extract | 0.1-2% |
| Saccharide (e.g. glucose) | 0.5-5% |
| Sodium Acetate | 0.1-2% |
| Surfactant/emulsifier (e.g. Polysorbate 80) | 0.005-3% |
| Dipotassium hydrogen phosphate | 0.005-1% |
| Ammonium citrate | 0.005-1% |
| Magnesium sulfate | 0.001-1% |
| Manganese sulfate | 0.001-1% |
| Agar | 0.1-5% |
| Reverse osmosis (RO) Water | q.s |
| Total | 100.0% |

The protection insert may be in any form or shape, e.g. cube, disk, powder, etc.

In some examples, the surface of the insert is roughened, to an extent that facilitates the habitation of surface depressions by the growing bacteria, thereby to allow better attachment of the bacteriocin producing bacteria onto the insert. To this end, at times, the substrate holding the nutrients composition may be actively roughened at its surface, e.g. by mechanical abrasion techniques. The roughening is even further importance when the consumer goods are typically cooled (e.g. in storage) and thus, the roughening facilitates the attachment of the bacteriocin producing bacteria only the surface of the substrate.

In some examples, the protection insert is placed within a carrier device for facilitating the holding of the insert in proximity to the goods to be preserved, e.g. by adhering the carrier device to the goods or to the package holding the goods.

The carrier device may be an open tray-like device in which the insert is fixed, a perforated or otherwise non-sealed sachet, a net like structure or any other carrier configuration that while holding the insert in place, it also maintains the insert exposed to the environment surrounding the goods.

The carrier device may be from any biocompatible/bio-acceptable material and there is a wide variety of such available material and especially relevant are those known to be used in the food industry, e.g. in the packaging industry. For example, the carrier may be formed from paper (cellulose based material), aluminum, rubber, plastic (e.g. polyethylene, polyprophylene), polystyrene and the like.

In some examples, the carrier device may have roughed sections on its surface exposed to the insert so as to facilitate desired bacteria growth thereon.

The insert can be incorporated with various consumer goods such as food stuff. Thus, also disclosed herein is a package comprising the goods to be preserved and the protection insert disclosed herein as well as a method of protecting good, the method comprising packaging said good in a package holding in vicinity with said good a protection insert disclosed herein.

In some examples, the goods to be preserved/protected comprise food.

In some examples, the food to be preserved/protected is a meat product, including marine animal meat, red meat, poultry, as well as vegan alternatives to meat.

In some examples, the food to be protected is harvested crop, e.g. fruits, vegetables, seeds and grains, as well as already pealed fruits and vegetables.

In some examples, the food to be preserved/protected is dairy or vegan alternatives to dairy products.

In some examples, the insert is packed with ready to cook or ready to eat food, such as pre-cooked meals, salads, etc.

In some examples, the protection insert is packed with consumer goods that have a high-water activity and thus more vulnerable to microbial spoilage. Examples of such products include but are not limited to fresh produce, food products such as cheese, meat, salads, spreads, cosmetics and toiletries, home care cleaning products.

In some examples, the insert is packed with consumer goods that otherwise would require storage within a refrigerator. In the presence of the protection insert such products can be stored at room temperature. Examples of such products may include, without being limited thereto, salad spreads, meat, fish, cheese, fruit and vegetables, pre peeled and pre-cut fruits and vegetables

In some examples, the goods include crops. The protection insert can be incorporated in the soil and stimulates plant growth promoting bacteria which further protect the plants from disease.

In some examples, the goods include home care products such as cleansing solutions which are normally preserved with chemicals.

In some examples, the goods include drugs in the form of an ointment, gel, cream, lotion or a solution, which are normally preserved with chemicals.

### DETAILED DESCRIPTION OF NON-LIMITING EMBODIMENTS

### EXAMPLE 1

### Food Protection Insert Compositions A food protection insert was prepared according to Table 2:

TABLE 2
Protection insert composition

| | |
|---|---|
| Ingredient | Amount (w %) |

Substrate forming material

| | |
|---|---|
| Agar | 1.124% |

Nitrogen containing compounds/source

| | |
|---|---|
| Vegetable Peptone | 0.937% |
| Yeast extract | 0.469% |
| Soy extract | 0.937% |

Carbohydrates

| | |
|---|---|
| Glucose | 1.874% |
| Fructooligosaccharides (FOS) | 2.000% |

Fatty acids

| | |
|---|---|
| Polysorbate 80 | 0.094% |

Inorganic minerals/Inorganic salts

| | |
|---|---|
| Sodium Acetate | 0.469% |
| Dipotassium hydrogen phosphate | 0.187% |
| Tri-ammonium citrate | 0.187% |
| Magnesium sulfate | 0.009% |
| Manganese sulfate | 0.005% |

Vitamins

| | |
|---|---|
| Calcium Panthenoate | 2.5 × 10⁻⁶% |
| Niacin | 2.5 × 10⁻⁶% |

Protection additive

| | |
|---|---|
| Potassium Sorbate | 0.0700% |

Buffering agent

| | |
|---|---|
| Citric Acid | q.s (pH 6-6.6) |

Water

| | |
|---|---|
| RO Water | q.s |
| Total | 100.0% |

The nutrient insert was prepared by mixing all ingredients and suspending the same in the reverse osmosis water to form a mixture. Then the mixture was heated under stirring, until reaching 100° C. The mixture was allowed to boil for 1-2 minutes to ensure complete dissolution, after which the mixture was cooled to 50° C. while thoroughly mixing and poured into the carrier device in quantities between 0.5-25 gr under aseptic conditions. Once in the carrier device, the insert composition was allowed to cool to room temperature.

### EXAMPLE 2

### Preservation of Foodstuff Using the Food Protection Insert

### EXAMPLE 2A

### Preservation of Cucumbers

A plastic open tube carrying the nutrient insert of Table 2 was placed inside a bag of cucumbers. As control, cucumbers bag without the insert was used.

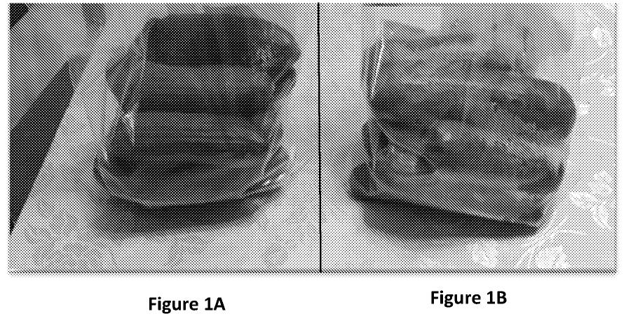

The treated bag (including the protection insert) and the control bag were both kept at an ambient environment (room temperature). FIGS. 1A-1B show the two bags after 12 days with the left bag containing the protection insert (“Preserved”, FIG. 1A) and a negative control bag containing the same weight of cucumbers but without the insert (“Control”, FIG. 1B).

The protection insert prolongs the shelf life of the cucumbers by at least 7 days, as evident by the lack of molds development. Without being bound by theory, it is believed that the lack of mold development was a result of growth of Bacillus Pumilis (data not shown) on the insert substrate, stimulated by the insert composition.

### EXAMPLE 2B

### Preservation of Strawberries

A plastic tube carrying the nutrient insert of Table 2 was attached to the inside of a strawberries bag by adhesive label. As control, a bag of strawberries without the insert was used.

The two bags were kept at room temperature for a period of 12 days.

The insert prolonged the microbial shelf life of strawberries by at least 3 days, as evident by the absence of yeast development as detected by the characteristic smell of ethyl acetate. In a separate experiment which was conducted at The Institute for food microbiology Haifa, it was shown that indeed the insert inhibited yeast growth. Without being bound by theory, it is believed that the lack of mold on the fruit was a result of growth of Bacillus Subtilis on the substrate, stimulated by the insert's nutrients composition.

### EXAMPLE 2C

### Perseveration of Tahini Spread

A sachet containing the protection insert of Table 2 was placed inside a pack of Tahini spread with no preservatives. The sachet was attached to the lid with an adhesive label.

The insert prolongs shelf life of the spread from 3 days in refrigeration to 5 days in room temperature. Without being bound by theory, it is believed that the stability of the spread is a result of growth of Bacillus Substilis on the substrate.

### EXAMPLE 3

### Additional Compositions for Use in a Protection Insert

In a similar manner to Example 1 additional compositions were prepared with different levels of efficiency and these are provided in Tables 3A-3B.

TABLE 3A
Additional insert compositions

| Component | Concentration % | | | | | | |
|---|---|---|---|---|---|---|---|
| No. | I | II | III | IV | V | VI | VII |
| Peptone | 6.57 | 6.57 | 6.57 | 6.57 | 6.57 | 6.57 | 6.57 |
| Beef extract | 0.57 | 0.57 | 0.57 | 0.57 | 0.57 | 0.57 | 0.57 |
| Yeast extract | 0.29 | 0.29 | 0.29 | 0.29 | 0.29 | 0.29 | 0.29 |
| Glucose | 1.14 | 1.14 | 1.14 | 1.14 | 1.14 | 1.14 | 1.14 |
| Sodium Acetate | 0.29 | 0.29 | 0.29 | 0.29 | 0.29 | 0.29 | 0.29 |
| Polysorbate 80 | 0.06 | 0.06 | 0.06 | 0.06 | 0.06 | 0.06 | 0.06 |
| Dipotassium hydrogen phosphate | 0.11 | 0.11 | 0.11 | 0.11 | 0.11 | 0.11 | 0.11 |
| Tri-ammonium citrate | 0.11 | 0.11 | 0.11 | 0.11 | 0.11 | 0.11 | 0.11 |
| Magnesium sulfate | 0.01 | 0.01 | 0.01 | 0.01 | 0.01 | 0.01 | 0.01 |
| Manganese sulfate | 0.003 | 0.003 | 0.003 | 0.003 | 0.003 | 0.003 | 0.003 |
| Potassium Sorbate | 0 | 0 | 0 | 0.12 | 0.12 | 0.12 | 0.12 |
| Citric Acid | 0 | 0 | 0 | 0.001 | 0.001 | 0.001 | 0.001 |
| Agar | 1.37 | 0.86 | 0.86 | 0.86 | 0.86 | 0.86 | 0.86 |
| Calcium Panthenoate | 0 | 0 | 0 | 0 | 0.008 | 0.008 | 0.008 |
| Niacin | 0 | 0 | 0 | 0 | 0 | 0.008 | 0.008 |
| Glycine | 0 | 0 | 0 | 0 | 0 | 0 | 1 |
| Water | 95.477 | 95.987 | 95.987 | 95.866 | 95.858 | 95.85 | 94.85 |
| Total | 100 | 100 | 100 | 100 | 100 | 100 | 100 |
| Final PH | 6.5 | 6.5 | 6.5 | 5.8 | 5.8 | 5.8 | 5.8 |
| Surface treatment | No | No | Yes | Yes | Yes | Yes | Yes |

TABLE 3B
observations from additional insert compositions

| No. | Observations |
|---|---|
| I | The composition is lacking some ingredients, including the |
| | preservative potassium sorbate, Niacin, and glycine and the pH |
| | was above 6. The growth of Bacillus was relatively slow (7-10 |
| | days @25° C.). |
| II | The composition includes a lower amount of agar as compared to |
| | composition I and shows a slight increase in the growth rate of |
| | Bacillus (5-7 days @25° C.) |
| III | Surface treatment (scratched to create a rough texture) of the gel |
| | improved Bacillus adherence to the nutrient culture and increased |
| | the growth rate (2-4 days @25° C.). However, the treatment |
| | impaired the selectivity of the composition and resulted also in the |
| | growth of yeast and enterococcus bacteria |
| IV | Addition of the preservative Potassium Sorbate combined with |
| | reducing pH to 5.8 inhibited growth of yeast and enterococcus |
| | bacteria (as compared to composition III); but also slightly affected |
| | the growth rate Bacillus growth. |
| V | Addition of calcium Panthenoate improved growth rate of Bacillus |
| | growth as compared to IV (5-7 days @25° C.) |
| VI | Addition of Niacin improved growth rate of Bacillus growth as |
| | compared to V (4-6 days @25° C.) |
| VII | Addition of Glycine improved growth rate of Bacillus growth as |
| | compared to VI (3-5 days @25° C.) |

### EXAMPLE 4

### Shelf Life Studies

The effect of the insert of Table 2 on shelf life prolongation is determined on pre-packed cucumbers. Specifically, 12 packs of cucumbers containing about 1 kg are divided into text groups,

Group I—6 packs pre-bagged with the insert of Table 2; the “Treated Group”.

Group II—6 packs without insert, the “Reference Group”.

Cucumber bags are be stored at 25° C., at ambient room humidity conditions for 14 days.

Samples of the Treated Group and Reference Group are analyzed at days 0, 7 and 14, using the following testing methods. Each test is repeated twice:
Deep sequencing (high-throughput sequencing).
Total counts using spread plate method on PCA.
Total Enterobacteriaceae counts.
Bacillus counts.
Lactic acid bacteria count.
Qualitative evaluation of visual appearance.

It is expected that the microbiome composition in the treated cucumbers will be different as compared to the reference and will contain more Bacillus and Lactic Acid bacteria species and lower levels of Enterobacteriacea and mold counts as compared to the control group. In other words, the results will show that the treatment disclosed herein enable longer shelf life for the cucumbers.

### EXAMPLE 5

### Soil Treatment

The effect of the insert of soil is also examined To this end, 4 planter boxes with fresh soil is used.

Test Group—2 planters each carrying the inset composition of Table 2 (2 inserts per 1 litter of soil).

Group I—2 planters with 1 litter of soil, each carrying the inset composition of Table 2; the “Treated Group”

Group II—0 2 planters with 1 litter of soil, without the insert; the “Reference Group”.

Each planter is planted with basil seeds.

The planter boxes are placed in a designated greenhouse for 21 days from planting.

Soil samples from the Treated Group and Reference Group are analyzed, on day 0 and 21, using the following testing methods:
Deep sequencing (high-throughput sequencing).
Total counts using spread plate method on PCA.
Total Enterobacteriaceae counts.
Bacillus counts.
Lactic acid bacteria count.
Qualitative evaluation of visual appearance.

It is expected that the microbiome composition in the treated soil will be different as compared to the reference and will contain more Bacillus and Lactic Acid bacteria species and lower levels of Enterobacteriacea and mold counts as compared to the control group. In other words, the results will show that the treatment of the soil disclosed herein improve growth of the plant.

1. A protection insert, comprising:
a discrete substrate-forming material holding a nutrient composition;
wherein the nutrient composition includes a combination of nutrients for supporting selective growth of one or more bacteriocin-producing bacterium on said substrate and being essentially free of externally added microorganisms.

2. The protection insert of claim 1, in a solid or semi solid form, suitable for placement in vicinity of a goods product that is in predisposition of becoming spoiled.

3. The protection insert of claim 1, wherein said substrate forming material includes at least one material selected from the group consisting of agar, guar gum, xanthan gum, gellan gum, carrageenan, gelatin, dextrin, and starch.

4. The protection insert of claim 1, wherein said nutrient composition comprises one or more of nitrogen containing compounds, carbohydrates, inorganic minerals and salts, fatty acids, and vitamins.

5. The protection insert of claim 1, wherein the nutrient composition comprises at least nitrogen containing compounds.

6. (canceled)

7. The protection insert of claim 5, wherein a source of said nitrogen containing compounds is any one or combination of animal extract, microorganism extract, and plant extract.

8. The protection insert of claim 1, wherein the nutrient composition comprises one or more carbohydrate.

9. The protection insert of claim 8, wherein said carbohydrates comprise any one of monosaccharides, disaccharides, oligosaccharides, or combination of same.

10. (canceled)

11. The protection insert of claim 1, wherein said nutrient composition comprises inorganic minerals and salts.

12. (canceled)

13. The protection insert of claim 1, wherein said nutrient composition comprises fatty acids.

14. (canceled)

15. The protection insert of claim 1, wherein said nutrient composition comprises at least one vitamin.

16. (canceled)

17. The protection insert of claim 1, wherein said nutrient composition comprises a buffering agent.

18. (canceled)

19. The protection insert of claim 1, having a pH in a range of 5.5 and 7.

20. The protection insert of claim 1, further comprising a solid carrier device for holding the discrete substrate-forming material at least partially exposed to the goods surroundings.

21. A consumer goods package comprising goods to be preserved and the protection insert as defined in claim 1.

22. The consumer goods package of claim 21, wherein said goods comprises vegetables or fruits.

23. The consumer goods package of claim 21, having a shelf life that is extended by at least 10% as compared to the same goods, when the goods are packed and stored under the same conditions, without the protection insert.

24. A consumer goods package comprising a planter holding soil, and the protection insert according to claim 1 within the soil.

25. A method of preserving consumer goods, the method comprising holding said goods in vicinity with said protection insert according to claim 1.

26. (canceled)
